(19) United States
(12) Patent Application Publication
(10) Pub. No.: US 2023/0210404 A1
(43) Pub. Date: Jul. 6, 2023

(54) SENSOR MODULE, CARE SET AND USE THEREFOR

(71) Applicant: MOIO GmbH, Fürth (DE)

(72) Inventors: Jürgen Besser, Tuchenbach (DE); Günther Maximilian Hefner, Riemerling (DE)

(21) Appl. No.: 17/608,960

(22) PCT Filed: May 6, 2020
(86) PCT No.: PCT/EP2020/062569

§ 371 Date: Mar. 31, 2022

(30) Foreign Application Priority Data
May 9, 2019 (DE) 10 2019 112 126.4

(51) Int. Cl. A61B 5/11; A61B 5/277

(52) U.S. Cl. CPC A61B 5/1112; A61B 5/277; A61B 2560/0443; A61B 2562/0219

(57) ABSTRACT

The invention relates to a sensor module (10) comprising a first submodule (101) comprising an electronic unit (105) for measuring and transmitting movement data, and a first structure (103), and a second submodule (201) comprising a fastening means for fastening the second submodule (201) to a wearer, and a second structure (205), wherein the first structure (103) can form a reversible mechanical connection with the second structure (205), with the result that the first submodule (101) and the second submodule (201) are reversibly connected to one another.

### BACKGROUND OF THE INVENTION

### Field of the Invention

The invention relates to a sensor module, a care set and the use of a submodule for capturing and evaluating health-related data of people in need of care.

### Prior Art

DE 10 2017 107 864 A1 discloses a skin patch which is intended to be releasably connected to the skin of a wearer. An adhesive layer is provided for this purpose on the side facing the skin. A pocket section in which an assistance system having electronic components can be accommodated is provided on the side facing away from the skin.

### SUMMARY OF THE INVENTION

The object of the present invention is to specify an apparatus which is improved in comparison with the prior art.

This object is achieved by means of a sensor module having the features of claim 1, a care set having the features of claim 14 and the use of a second submodule having the features of claim 15.

In a first aspect, the invention presents a sensor module comprising a first submodule with a first structure and with an electronic unit for capturing and transmitting movement data. The sensor module according to the invention also comprises a second submodule with a fastening means for fastening the second submodule to a wearer, and a second structure, wherein the first structure can form a reversible mechanical connection with the second structure, with the result that the first submodule and the second submodule can be reversibly connected to one another.

This advantageously achieves the situation in which the first submodule containing the electronic unit can be removed from the wearer without the second submodule, which is fastened to the wearer by the fastening means, having to be removed from the wearer. This achieves additional flexibility. For any replacement or repair of the first submodule or parts of the latter, for example the electronic unit, it is not necessary to remove the entire sensor module from the wearer. This is also more pleasant for the wearer to whom the second submodule is fastened since the fastening need not be removed and attached again. Rather, it is sufficient to release the reversible connection between the first structure of the first submodule and the second structure of the second submodule in order to separate the first and second submodules from one another.

In one advantageous embodiment, the first structure comprises one from the group of hook structure or loop structure and the second structure comprises the other from the group of hook structure or loop structure, which structures form a reversible mechanical connection when the first structure is pressed together with the second structure. The reversible mechanical connection is preferably in the form of a reversible form fit, in particular a hook-and-loop fastening. This ensures a reliable releasable fastening which releasably connects the first submodule to the second submodule.

In a further advantageous embodiment, the fastening means comprises an adhesive layer, in particular an adhesive layer which can adhere to the human skin. This has the advantage that the sensor module can be directly attached to a skin region of a wearer with a material bond. As a result of the fact that the first submodule and the second submodule are connected by means of a reversible mechanical connection, it is not necessary, for the purpose of removing the first submodule, to release the adhesive layer which is used to fasten the second submodule to the wearer's body. Avoiding the release and reattachment of the adhesive layer also results in a longer adhesive effect of the adhesive layer, and the adhesive layer and the second submodule can each be renewed after a longer interval of time. This is also advantageous for the wearer who has to endure the removal of the adhesive layer less often.

The second submodule expediently comprises a stretchy adhesive strip. As a result, the second submodule can be optimally adapted to the geometry of the skin surface to which the second submodule is adhesively bonded. This is particularly advantageous if the wearer is moving and the geometry of the skin surface changes dynamically.

The second submodule preferably comprises an abdominal bandage. This makes it possible to fasten the second submodule to the wearer, which fastening is mechanically releasable and does not require an adhesive layer.

The electronic unit preferably comprises at least one acceleration sensor for measuring acceleration data. The measured acceleration data are used to evaluate the movement of the wearer. This can comprise the detection of a fall, for example. It is also possible to evaluate the data with respect to stretcher detection, position detection and decubitus detection.

Alternatively or additionally, the electronic unit comprises a GPS module for capturing and transmitting spatial coordinates of the first submodule or of the sensor module, wherein GPS stands for Global Positioning System. This makes it possible to locate and analyze the activity of the wearer. This is advantageous if, for example, the evaluation of the acceleration data has revealed a fall of the wearer and the location of the wearer is intended to be determined as quickly as possible.

In a further advantageous embodiment, the second submodule comprises a near-field communication transmitter for transmitting data, and the first submodule comprises a near-field communication receiver for receiving the data, wherein the data are used to verify
a) the functionality of the pairing of the first submodule with the second submodule, and/or
b) the correct attachment of the first submodule, and/or
c) the use of a proprietary second submodule.

Data interchange in near-field communication, for example via RFID which means Radio Frequency Identification, is carried out using electromagnetic induction and is therefore possible only at short ranges of a few centimeters to approximately 10 cm. This short range is used in the present case to verify the pairing in order to determine whether the second submodule is arranged in the local vicinity of the first submodule or is connected to the latter. The verification of the correct attachment ensures that the sensor module is arranged correctly with respect to the front and rear sides/top side and underside, that is to say the first submodule is fastened to the second submodule with the correct alignment with the latter. Furthermore, transmitting an ID signal ensures that a verified original submodule is used as the second submodule. These verifying steps ensure reliable operation of the sensor module and the correctness of the data to be captured and transmitted. In addition, the verification steps mentioned are necessary prerequisites for activating the functions of the first submodule.

The first submodule preferably comprises a capacitive sensor for measuring capacitance values between a capacitor electrode belonging to the capacitive sensor and the wearer's skin that acts as a second electrode. The capacitive sensor is preferably arranged on the electronic unit. However, the capacitive sensor may also be arranged at another position of the first submodule. In any case, the capacitive sensor is electrically connected to the electronic unit, with the result that the capacitance values measured by the capacitive sensor can be transmitted to the electronic unit. It is known that the capacitance of a capacitor is dependent on the distance between its electrodes. Measuring capacitance values using the present arrangement accordingly makes it possible to determine whether the first submodule has been separated from the second submodule which is fastened to the wearer. It is likewise possible to determine whether the sensor module has been removed from the wearer. This is because the capacitance also changes in this case.

It is advantageous if the electronic unit comprises a comparison unit which is designed to compare a capacitance reference value with a present capacitance value and to prompt data to be measured, captured and/or transmitted only when the present capacitance value is greater than or equal to the reference value. The data are preferably the data relating to the acceleration, the spatial coordinates and the verification. The reference value can be selected, for example, in such a manner that it correlates with the distance between the first submodule or the capacitor electrode and the wearer's skin surface if the sensor module is fastened to the wearer's skin surface. The comparison with this reference value makes it possible to reliably determine whether the first submodule has been separated from the second submodule which is fastened to the wearer or whether the sensor module has been removed from the wearer.

It is also possible for the data to be measured, captured and/or transmitted only when the present capacitance value is less than or equal to the reference value.

In a further advantageous embodiment, the electronic unit comprises a communication module which is used to transmit the captured data to an evaluation unit outside the sensor module. This enables the targeted evaluation and inspection of the data on an associated evaluation unit by an appropriate expert. The communication module may be a GSM communication module, in which case GSM means a global mobile radio standard.

The first submodule expediently comprises a flexible conductor connection which connects the electronic unit to a power source which supplies the electronic unit with energy. This has the advantage that the conductor connection can adapt to the movements of the wearer, provided that the sensor module is accordingly attached to the wearer. The sensor module is advantageously attached to the wearer's back in such a manner that the conductor connection runs parallel or perpendicular to the spinal column in order to adapt to conventional movements such as bending forward.

The first submodule preferably comprises a sheath made of a flexible material which preferably encloses the power source and the electronic unit by means of a molding method, with the result that the power source and the electronic unit form a non-releasable form fit with the sheath. As a result, the power source and the electronic unit are protected from mechanical influences and detachment of the power source and/or the electronic unit from the sheath is also prevented.

The sheath expediently comprises at least one flexible, preferably linear, region, about which the first submodule can be bent, which region forms a bending axis, about which the first submodule can be bent, with the result that the flexible conductor connection is likewise bent when bending the first submodule around the linear region. This has the advantage that the first submodule can adapt during use to the movements of the wearer wearing the sensor module.

A second aspect of the invention presents a care set comprising a sensor module according to the invention and an evaluation unit which is arranged separately from the sensor module and has software which is used to receive, evaluate and display the measured data. Alternatively, the care set may also comprise a sensor module according to the invention and a computer program product which, when installed and executed on an electronic evaluation unit, forms software which is used to receive, evaluate and display the measured data.

The reception and evaluation are carried out by means of a separate evaluation unit having software by an expert who can initiate suitable measures, if appropriate, on the basis of the evaluated data. For example, it is necessary, if it is detected that the first submodule is no longer fastened to the second submodule because the near-field communication receiver no longer receives any data, to fasten the first submodule to the second submodule again.

The computer program product is preferably used in a computer, a smartphone or a tablet. The evaluation on a computer makes it possible to access further data relating to the wearer which are usually stored in a secure network. The newly generated data can also be directly added to the data record for the respective wearer. The use of mobile devices such as smartphones or tablets as an evaluation unit has the advantage that the data and the evaluation can be processed promptly. This is because a mobile device is usually carried continuously, with the result that, for example, an acoustic signal indicating that an important change has arisen in a wearer wearing the sensor module can directly result in a necessary measure. This may relate to the separation of the first submodule from the second submodule, but may also relate to the detection of a fall which has been detected by evaluating the acceleration data.

A third aspect of the invention presents the use of a second submodule for connection to a first submodule in a sensor module, wherein the first submodule can capture and transmit movement data by means of an electronic unit and comprises a first structure, and wherein the second submodule comprises a fastening means for fastening to a wearer, and comprises a second structure which forms a reversible mechanical connection with the first structure when being pressed together, with the result that the first submodule and the second submodule are reversibly connected to one another.

Use is preferably made of a near-field communication transmitter which is suitable for transmitting data to a near-field communication receiver, wherein the data are used to verify
a) the functionality of the pairing of the first submodule with the second submodule, and/or
b) the correct attachment of the first submodule, and/or
c) the use of a proprietary second submodule.

### BRIEF DESCRIPTION OF THE DRAWINGS

Further features and advantages of the invention emerge from the following description of preferred exemplary embodiments of the invention on the basis of the drawings, in which:

FIG. 1A schematically shows a first front side of a first submodule of the sensor module according to the invention.

FIG. 1B schematically shows a first rear side of the first submodule of the sensor module according to the invention.

FIG. 2A schematically shows a second front side of a second submodule of the sensor module according to the invention.

FIG. 2B schematically shows a second rear side of the second submodule of the sensor module according to the invention.

FIG. 3 schematically shows the sensor module according to the invention adhesively bonded to the back of a wearer, and an evaluation unit.

### DESCRIPTION OF PREFERRED EXEMPLARY EMBODIMENTS

In the following description of preferred embodiments of the present invention, identical reference signs denote identical or comparable components.

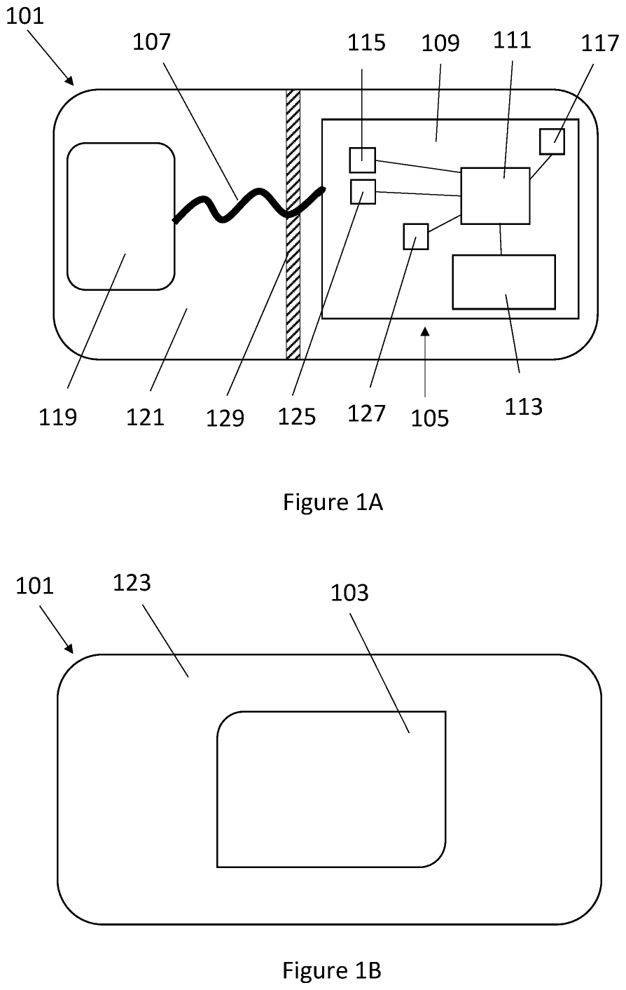

FIG. 1A shows a first front side 121 of a first submodule 101 of the sensor module 10 according to the invention. The first submodule 101 is flat and has a rectangular base with rounded corners which may also be not rounded. The base may likewise be square or have another shape. An electronic unit 105 is arranged on the first front side 121 and comprises a circuit board 109 having electronic components 111, 113, 115, 117, 125, 127 which are arranged on the latter and are electrically connected to one another. The circuit board 109 is connected, via a flexible electrical line 107, to a power source 119, for example a battery, which supplies the circuit board 109 and the electronic components 111, 113, 115, 117, 125, 127 arranged on the latter with energy. The circuit board 109 may also be in the form of a printed circuit board. The electronic components arranged on the circuit board 109 comprise one or more acceleration sensors 115, a microcontroller 111, a communication module 113, a near-field communication receiver 117, a capacitive sensor 127 and a GPS module 125, where GPS means Global Positioning System. The communication module 113 may be in the form of a GSM communication module, where GSM means a global mobile radio standard. The capacitive sensor 127 also has a capacitor electrode. In the assembled state of the sensor module 10, that is to say when the first submodule 101 is fastened to the second submodule 201 and the second submodule 201 is fastened to the wearer's skin surface, a capacitance can be accordingly measured between the capacitor electrode and the wearer's skin surface since the wearer's skin acts as an electrode. The capacitance measured in this assembled state can be used as a reference value for further measurements. The electronic unit 105 comprises a comparison unit (not illustrated) which is designed to compare this capacitance reference value with a present capacitance value. If the present capacitance value is greater than or equal to the reference value, the comparison unit accordingly prompts the data relating to the acceleration, the spatial coordinates and the verification to be measured, captured and/or transmitted. If the present capacitance value is less than the reference value, that is to say the first submodule 101 has been removed from the second submodule 201 or the entire sensor module 10 has been removed from the wearer's skin surface, no data relating to the acceleration, the spatial coordinates and the verification are measured, captured and/or transmitted. In this situation, the electronic unit 105 can change to a quiescent state in which the capacitance values are measured only in a desired interval and are compared with the reference value. As soon as the presently measured capacitance value is greater than or equal to the reference value again, the data relating to the acceleration, the spatial coordinates and the verification are measured, captured and/or transmitted.

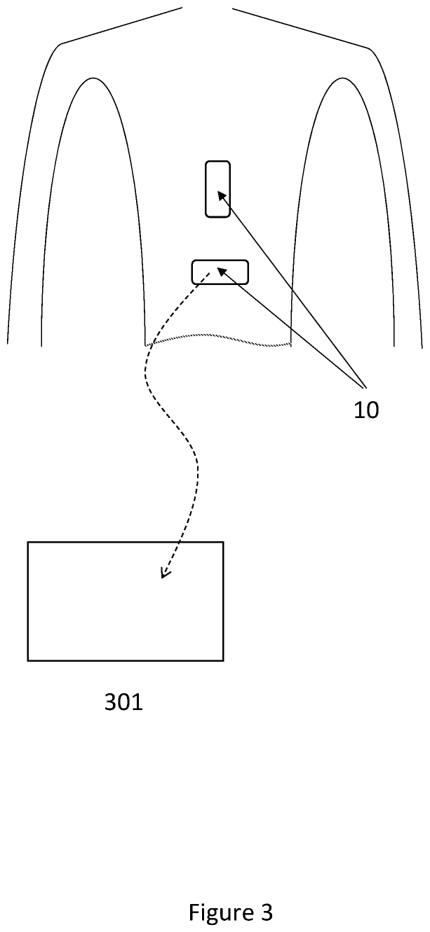

Acceleration data are measured using the one or more acceleration sensors 115 and are accordingly forwarded to the microcontroller 111. The use of a plurality of acceleration sensors 115 makes it possible to measure accelerations in different directions. The microcontroller 111 forwards the data received from the at least one acceleration sensor 115, the GPS module and the near-field communication receiver 117 to the communication module 113 which transmits the data to an external evaluation unit 301 (see FIG. 3). The capacitances measured by the capacitive sensor are likewise forwarded to the evaluation unit 301 via the communication module 113.

The first submodule 101 is surrounded by a silicone sheath. The silicone sheath and the silicone structure on the first front side 121 can be produced using injection-molding technology. In this case, the power source 119 and the electronic unit 105 are enclosed by the silicone sheath. In the case of a rectangular base having a longitudinal axis and a transverse axis, the power source 119 and the electronic unit 105 are embedded at a distance beside one another along the longitudinal axis of the rectangle. A mechanically flexible, preferably linear, region 129, for example in the form of a depression in the material, is formed between the electronic unit 105 and the power source 119 transversely with respect to the longitudinal axis of the rectangle. As a result, the first submodule 101 is mechanically flexible along the transverse axis of the rectangle, which runs between the arranged power source 119 and the circuit board 109, and can adapt to the movements of the wearer while being worn by the latter. The mechanically flexible region 129 is formed on the first front side 121 in the present exemplary embodiment. The mechanically flexible region 129 may likewise be formed on the first rear side 123 or both on the first front side 121 and on the first rear side 123 in a manner opposite one another.

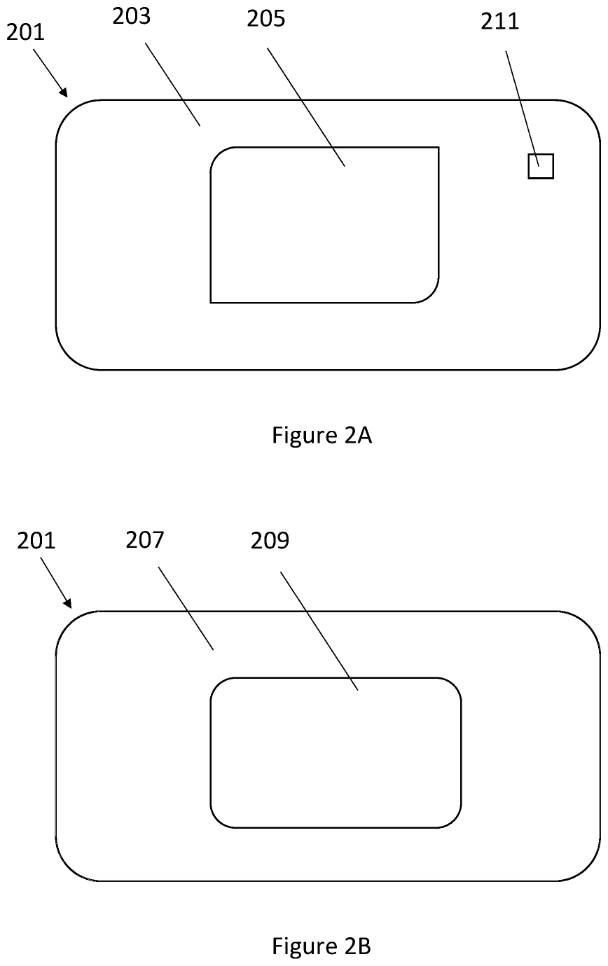

The near-field communication receiver 117 is suitable for receiving the transmitted data from a near-field communication transmitter 211 which is arranged on the second submodule 201 (see FIG. 2A). The near-field communication receiver 117 and the near-field communication transmitter 211 communicate on the basis of the near-field communication standard (NFC standard) RFID which means Radio Frequency Identification. Data interchange in near-field communication is carried out using electromagnetic induction and is therefore possible only at short ranges of a few centimeters to approximately 10 cm.

FIG. 1B shows a first rear side 123 of the first submodule 101 which faces away from the first front side 121. The first rear side 123 comprises a first structure 103 which may be in the form of a hook or loop structure. The first structure 103 may extend over a section of the first rear side 123 or over the entire first rear side 123.

FIG. 2A shows a second front side 203 of a second submodule 201. A second structure 205 which is in the form of a hook or loop structure and forms the counterpart to the hook or loop structure of the first structure 103 of the first submodule 101 is formed on the second front side 203. When the first structure 103 is pushed together with the second structure 203, the hook and loop structures accordingly connect and a mechanically reversible form-fitting connection is formed, for example a hook-and-loop fastening. As a result, the first submodule 101 and the second submodule 201 are connected to one another by means of a mechanical reversible form fit. The second structure 203 may extend over a section of the second front side 203 or over the entire second front side 203.

The mechanically reversible form-fitting connection makes it possible to remove the first submodule 101 for possible modifications or repairs, while the second submodule 201 remains adhering at the respective position on the body of a wearer by virtue of the adhesive bond 209. This modular structure also makes it possible for the first submodule 101 to be able to be replaced by another submodule.

A near-field communication transmitter 211 is also arranged on the second front side 203. The near-field communication transmitter 211 may also be arranged on the second rear side 207 or may be integrated in the second submodule 201, with the result that the near-field communication transmitter 211 is not visible to the outside. For successful data transmission, it is necessary for the near-field communication transmitter 211 to be arranged in the local vicinity of the near-field communication receiver 117, that is to say at a distance of less than 10 cm. The data which are transmitted by the near-field communication transmitter 211 to the near-field communication receiver 117 can be verified with respect to the functionality of the pairing of the first submodule 101 with the second submodule 201, the correct attachment of the first submodule 101 to the second submodule 201 with regard to the front and rear sides and the alignment, and the use of a proprietary second submodule 201. The presence of a functioning pairing can be verified via the functionality of near-field communication per se. If the first submodule 101 is too far away from the second submodule 201, that is to say more than 10-15 cm for example, and near-field communication is no longer possible, pairing cannot be carried out either. The same applies if the first submodule 101 is not correctly connected to the second submodule 201, that is to say if the bases of the two submodules 101, 201 have been rotated through 180° with respect to one another. In this case, although the first submodule 101 and the second submodule 201 would be connected via the mechanically reversible connection, the near-field communication transmitter 211 would not be arranged in the immediate vicinity of the near-field communication receiver 117, with the result that no near-field communication can take place. Furthermore, the data transmitted by the near-field communication transmitter 211 may contain an identification signal in order to ensure that the second submodule 201 is an original submodule and is not a replica.

Additionally or alternatively, it is also possible to couple the determination of the capacitance reference value to successful pairing. As soon as pairing has been carried out by the near-field communication transmitter 211 and the near-field communication receiver 117, a first capacitance measurement by the capacitive sensor is initiated and can be used as the capacitance reference value.

FIG. 2B shows a second rear side 207 of the second submodule 201 which faces away from the second front side 203. The second rear side 207 comprises an adhesive layer 209 which remains adhering to the human skin and is used to fasten the second submodule 201 to the wearer's body. The adhesive layer 209 may extend over a region of the second rear side 207 or over the entire second rear side 207. The adhesive layer may be in the form of a biocompatible stretchy plaster adhesive strip.

FIG. 3 shows the sensor module 10 according to the invention, for example attached at two different positions on the back of a person. However, it is also possible to attach the sensor module 10 at other points of the back or to the chest or in the abdominal region. The sensor module 10 is preferably arranged along the spinal column or at right angles to the latter, starting from one of the longitudinal axes of a rectangular base. This has the advantage that flexible apparatus components, that is to say the flexible conductor connection 107 and the flexible linear region 129 of the silicone sheath, can at least partially follow the wearer's movements.

A separately arranged evaluation unit 301 is also shown and can receive and evaluate data from the communication module 113 of the electronic unit 105. The data transmitted to the evaluation unit 301 via the communication module 113 are evaluated, on the basis of data from the acceleration sensors 115, the near-field communication transmitter 211 and the capacitive sensor, with respect to stretcher detection, fall detection, position detection and decubitus detection. Localization and activity analysis are also possible using data from the GPS module 125 which are likewise transmitted to the evaluation unit 301 via the communication module 113. The data are evaluated on a computer and/or a mobile device, for example a tablet or a smartphone. As a result, evaluation is carried out in a connected infrastructure and necessary measures can be taken promptly, if appropriate. In addition, the data for the respective wearer can be centrally stored by a computer which is connected to a network protected from access by third parties. The evaluation of the data on a mobile device has the advantage that the information relating to an incident, for example a fall, is immediately detected since a mobile device is usually carried by the user, that is to say an authorized expert.

The sensor module 10 according to the invention and the care set are preferably used in facilities for people in need of care, such as care homes or nursing homes in which the people in need of care wear a respective sensor module 10 and care experts evaluate the captured data.

### LIST OF REFERENCE SIGNS

10 Sensor module
101 First submodule
103 First structure
105 Electronic unit
107 Flexible conductor connection
109 Circuit board
111 Microcontroller
113 Communication module
115 Acceleration sensor
117 Near-field communication receiver
119 Power source
121 First front side
123 First rear side
125 GPS module
127 Capacitive sensor
129 Flexible region
201 Second submodule
203 Second front side
205 Second structure
207 Second rear side
209 Adhesive layer
211 Near-field communication transmitter
301 Evaluation unit

1. A sensor module (10) comprising
a first submodule (101) comprising
a) an electronic unit (105) for capturing and transmitting movement data, and
b) a first structure (103), and
a second submodule (201) comprising
a) a fastening means for fastening the second submodule (201) to a wearer, and
b) a second structure (205),
characterized in that
the first structure (103) can form a reversible mechanical connection with the second structure (205), with the result that the first submodule (101) and the second submodule (201) can be reversibly connected to one another.

2. The sensor module (10) as claimed in claim 1, wherein the first structure (103) comprises one from the group of hook structure or loop structure and the second structure (205) comprises the other from the group of hook structure or loop structure, which structures form a reversible mechanical connection when the first structure (103) is pressed together with the second structure (205).

3. The sensor module (10) as claimed in either of claims 1 and 2, wherein the reversible mechanical connection comprises a reversible form fit, in particular a hook-and-loop fastening.

4. The sensor module (10) as claimed in one of claims 1 to 3, wherein the fastening means comprises an adhesive layer (209), in particular an adhesive layer (209) which can adhere to the human skin.

5. The sensor module (10) as claimed in one of claims 1 to 4, wherein the second submodule (201) is in the form of a stretchy adhesive strip.

6. The sensor module (10) as claimed in one of claims 1 to 3, wherein the second submodule (201) is in the form of an abdominal bandage.

7. The sensor module (10) as claimed in one of claims 1 to 6, wherein the electronic unit (105) comprises at least one acceleration sensor (115) for measuring acceleration data and/or a GPS module (125) for capturing and transmitting spatial coordinates of the first submodule (101) or of the sensor module (10).

8. The sensor module (10) as claimed in one of claims 1 to 7, wherein
the second submodule (201) comprises a near-field communication transmitter (211) for transmitting data, and
the first submodule (101) comprises a near-field communication receiver (117) for receiving the data, wherein
the data are used to verify
a) the functionality of the pairing of the first submodule (101) with the second submodule (201), and/or
b) the correct attachment of the first submodule (101), and/or
c) the use of a proprietary second submodule (201).

9. The sensor module (10) as claimed in one of claims 1 to 8, wherein the first submodule (101) comprises a capacitive sensor (127) for measuring capacitance values between a capacitor electrode belonging to the capacitive sensor (127) and the wearer's skin surface.

10. The sensor module (10) as claimed in one of claims 1 to 9, wherein the electronic unit (105) comprises a comparison unit which is designed to compare a capacitance reference value with a present capacitance value and to prompt data to be measured, captured and/or transmitted only when the present capacitance value is greater than or equal to or less than or equal to the reference value.

11. The sensor module (10) as claimed in one of claims 1 to 9, wherein the electronic unit (105) comprises a comparison unit which is designed to compare a capacitance reference value with a present capacitance value and to prompt the data relating to the acceleration, the spatial coordinates and the verification to be measured, captured and/or transmitted only when the present capacitance value is less than or equal to the reference value.

12. The sensor module (10) as claimed in one of claims 1 to 9, wherein the electronic unit (105) comprises a comparison unit which is designed to compare a capacitance reference value with a present capacitance value and to prompt the data relating to the acceleration, the spatial coordinates and the verification to be measured, captured and/or transmitted only when the present capacitance value is greater than or equal to the reference value.

13. The sensor module (10) as claimed in one of claims 1 to 12, wherein the electronic unit (105) comprises a communication module (113) which is used to transmit the captured data to an evaluation unit outside the sensor module (10).

14. The sensor module (10) as claimed in one of claims 1 to 13, wherein the first submodule (101) comprises a flexible conductor connection (107) which connects the electronic unit (105) to a power source (119) which supplies the electronic unit (105) with energy.

15. The sensor module (10) as claimed in one of claims 1 to 14, wherein the first submodule (101) comprises a sheath made of a flexible material which preferably encloses the power source (119) and the electronic unit (105) by means of a molding method, with the result that the power source (119) and the electronic unit (105) form a non-releasable form fit with the sheath.

16. The sensor module (10) as claimed in claim 15, wherein the sheath comprises at least one flexible, preferably linear, region (129) which forms a bending axis, about which the first submodule (101) can be bent, wherein the flexible region runs transversely with respect to the flexible conductor connection (107), with the result that the flexible conductor connection (107) is preferably likewise bent when bending the first submodule (101) around the linear region.

17. A care set comprising
a sensor module (10) as claimed in one of claims 1 to 16, and
a) an electronic evaluation unit (301) which is arranged separately from the sensor module (10) and has software which is used to receive, evaluate and display the measured data, or
b) a computer program product which, when installed and executed on an electronic evaluation unit, forms software which is used to receive, evaluate and display the measured data.

18. The use of a second submodule (201) for connection to a first submodule (101) in a sensor module (10), wherein the first submodule (101) can capture and transmit movement data by means of an electronic unit (105) and comprises a first structure (103),
wherein the second submodule (201)
a) comprises a fastening means for fastening to a wearer, and
b) comprises a second structure (205) which forms a reversible mechanical connection with the first structure (103) when being pressed together, with the result that the first submodule (101) and the second submodule (201) are reversibly connected to one another.

19. The use of the second submodule (201) as claimed in claim 18 which comprises a near-field communication transmitter (211) which is suitable for transmitting data to a near-field communication receiver (117), wherein the data are used to verify
a) the functionality of the pairing of the first submodule (101) with the second submodule (201), and/or
b) the correct attachment of the first submodule (101), and/or
c) the use of a proprietary second submodule (201).
